(19) United States
(12) Patent Application Publication
(10) Pub. No.: US 2012/0263798 A1
(43) Pub. Date: Oct. 18, 2012

(54) Body Aqua

(76) Inventor: Sherman Williams, JR., Summerville, SC (US)

(21) Appl. No.: 13/439,635

(22) Filed: Apr. 4, 2012

Related U.S. Application Data
(60) Provisional application No. 61/516,464, filed on Apr. 4, 2011.

(51) Int. Cl. A61K 33/30; A61K 33/32; A61K 33/42; A61K 33/22; A61P 25/00; A61P 17/00; A61Q 19/08; A61P 19/02; A61P 3/02; A61P 3/04; A61K 33/34; A61K 36/185

(52) U.S. Cl. 424/604; 424/643; 424/638; 424/639; 424/657; 424/776

(57) ABSTRACT

The invention provides an entire line of deliciously flavored bottled water beverages with each flavor offering comprised of vitamins, minerals and nutritional supplements for fortifying the body. A sugar free, carbohydrate free and low sodium formula, Body Aqua features natural flavoring and natural sweeteners such as Stevia™. The Body Aqua product line contains ten varieties, with each formula designed to target a specific area of the body and promote overall health.

### CLAIM OF PRIORITY

This patent application claims priority under 35 USC 119(e) (1) from U.S. Provisional Patent Application Ser. No. 61/516,464 filed Apr. 4, 2011, of common inventorship herewith entitled, “Body Aqua.”

### Field of the Invention

The present invention pertains to the field of health foods, and more specifically to the field of bottled beverages fortified with vitamins and minerals.

### BACKGROUND OF THE INVENTION

The prior art has put forth several designs for fortified beverages. Among these are:

U.S. Pat. No. 7,090,878 to Haile Mehansho, Renee Irvine Mellican, Raul Victorino Nunes, and Adrian Monsalve Marcano describes a water composition fortified with at least one mineral and has a pH balance between approximately 2.5 and 9.5. The mineral is selected from calcium, iron, zinc, copper, manganese, iodine, magnesium, and mixtures of these minerals. This water composition is free of flavor or sweetener compounds, has no metallic taste or aftertaste, and may contain optional vitamins and nutrients.

US Patent 2004/0219274 to Phillip Michael Cook describes a beverage composition that provides a water soluble source of Vitamin E while maintaining the beverage clarity. This invention can be used in fruit juices, carbonated and noncarbonated beverages. Various additives may be used in this composition such as flavoring agents, colorants, preservatives, sweeteners, vitamins, minerals and electrolytes.

U.S. Pat. No. 6,326,040 to Donald Raymond Kearney, Sanford Theodore Kirksey, Jr., and Donald Joseph Cox describes beverage products with improved vitamin stability. These beverage products include an effective amount of an aminopolycarboxylic acid, an effective amount of polyphosphate, and an enrichment with one or more vitamins or other nutrients. This beverage includes added water, fruit juice, ascorbic acid, ethylene diamine tetra acetic acid EDTA, sodium hexametaphosphate, and potassium sorbate. The beverage is contained in HDPE type containers which are oxygen permeable, eliminating the need for special oxygen barrier packaging.

None of these prior art references describe the present invention.

### SUMMARY OF THE INVENTION

It is an object of the present invention to provide a nutritionally fortified, sugar free, low calorie, carbohydrate free and low sodium beverage.

### DETAILED DESCRIPTION OF THE INVENTION

Vitamins and minerals are substances the body needs in small amounts for normal growth, function, and health. Essential for body functions, mental alertness, and resistance to infection, vitamins enable the body to process proteins, carbohydrates, and fats. Certain vitamins help produce blood cells, hormones, genetic material, and chemicals in the nervous system. Carbohydrates, proteins, and fat provide fuel and calories, while vitamins and minerals assist the body in releasing and using calories from food. Since these micronutrients are not created by the body naturally, they must be taken from food or supplements. Vitamins, minerals and other nutritional supplements are offered in pill form and taken by millions of children and adults every day. Many medicine chests and kitchen cabinets are filled with bottles of vitamin supplements. Vitamin A is for healthy vision, bone growth, and reproduction. Calcium promotes strong teeth and bones and efficient muscle and nerve function. Vitamin C maintains skin integrity and fortifies the immune system. While nutritional supplements are important for optimal health, many consumers attest that it is a daunting challenge to keep up with a plethora of supplements and remember the recommended daily dosage for each one. These pills often tend to be quite large and hard to swallow, and also leave an unpleasant taste in one's mouth. Many health conscious consumers miss a needed vitamin, or decide the effort is too overwhelming or unappealing and opt out of taking vitamins at all.

The present invention, hereinafter referred to as Body Aqua, is a practical solution to the aforementioned problems. Body Aqua is an entire line of deliciously flavored bottled water beverages with each flavor offering comprised of vitamins, minerals and nutritional supplements for fortifying the body. A sugar free, carbohydrate free and low sodium formula, Body Aqua features natural flavoring and natural sweeteners such as Stevia™. The Body Aqua product line contains multiple varieties, with each formula designed to target a specific area of the body and promote overall health. The list of the various formulas offered in the Body Aqua line, as well as a basic ingredient listing, are described in the following paragraphs.

The Cardio/Energy Igniter Fuel Formula offers the existing kinetic and potential energy of water with nutritional supplements, in a delicious naturally sweetened fruity punch flavor. These supplements provide an energy boost which is improved upon when the user exercises or increases their activity level. Key ingredients in this formula include glutamine, alanine, arginine, BCAAS, Vitamin C, Vitamin B complex, biotin, iron, magnesium, zinc and ginseng.

### Cardio/Energy/Igniter Fuel Formula

Glutamine—(3 G)
Alanine—(10 MG)
Arginine—(10 G)
BCAA's (Branch chain amino acids)—(4.8 G)
Vitamin C—(2000 MG, & B complex—100 MG)
Biotin—(100 MCG)
Iron—(15 MG)
Magnesium—(400 MG)
Zinc—(0.5 MG)
Ginseng—(800 MG)

The Skin/Wrinkles Formula utilizes the natural benefits of fresh lime juice to revitalize the skin. This flavored beverage minimizes fine lines and wrinkles, treats skin blotches, moistens dry and rough skin, and improves skin tone and elasticity. The key ingredients offered in this formula are Vitamin B complex, Vitamin A, Vitamin K, Vitamin E, Vitamin C, pyridoxine, biotin, selenium, copper, zinc, DMAE, chromium, alpha lipic acid and hyaluronic acid.

### Skin/Wrinkles Formula

Vitamin B/Complex (100 MG)
Pyridoxine (1 MG)
Biotin (30 MCG)
Vitamin A (5,000 IU), K (20 MCG), E (200 IU), C (250 MG)
Copper (1.5 MG)
Zinc (0.5 MG)
DMAE Dimethylaminoethanol (200 MG)
Chromium—(50 MG)
Alpha-biopic Acid (20 MG) Hyaloronic Acid (25 MG)

The Joints/Cartilage Formula is naturally flavored beverage, developed to provide the body with nutrients, which facilitate strong joints and cartilage lubrication and repair. The main ingredients in this formula are proline, gucosamine, fish oil, methyl sulfonylmethone, Vitamin A, Vitamin B, Vitamin C, Vitamin D, manganese, and omega 3 fatty acids.

### Joints/Cartilage Formula

Proline (500 MG)
Glucosamine (2.5 G)
Fish Oil (3 G)
Methyl Sulfonylmethone (MSM)—(1 G)
Vitamin A (5,000 IU), B (100 MG), C (250 MG), D (400 IU)
Manganese (400 MG)
Omega 3 Fatty Acids (1.5 G)

The All Essential Vitamin Mineral Formula, is an enriched formula of the Body Aqua which replenishes the natural resources needed to fuel the body and promote overall health. Two offerings are developed to meet the specific needs of the male or female body. The formula for men Contains Vitamin A, Vitamin B, Vitamin C, Vitamin E, riboflavin, niacin, pyridoxine, calcium, phosphorous, magnesium and zinc. The Vitamin Mineral formula for women contains Vitamin A. Vitamin C, Vitamin E, Vitamin D, Vitamin K, Vitamin B-6, Vitamin B-12, folic acid, calcium, boron and magnesium.

### Essential Vitamins/Minerals Formula

| Men | Women |
|---|---|
| Men Women | |
| Vitamin A (15,000IU) | Vitamin A (15,000IU) |
| Vitamin C (250 MG) | Vitamin C (250 MG) |
| Vitamin E (200IU) | Vitamin E (200IU) |
| Vitamin B (50 MG) | Vitamin D (400IU) |
| Riboflavin (18 MG) | Vitamin B-6 (100 MG), B-12 |
| | (100 MCG) |
| Niacin (1000 MG) | Folic Acid (600 MCG) |
| Pyridoxine (11 MG) | Calcium (800 MG) |
| Calcium (800 MG) | Boron (5 MG) |
| Phosphorous (800 MG) | Magnesium (400 MG) |
| Magnesium (400 MG) | Vitamin K (20 MCG) |
| Zinc (.5 MG) | |

The Weight Control/Belly Fat Formula, facilitates weight control while reducing fat in the belly area of the body. The key ingredients of the Weight Control/Belly Fat formula are Vitamin B-2, Vitamin B-3, Vitamin B-5, Vitamin B-6 and Vitamin C, choline, and inositol.

### Weight Control/Belly Fat Formula

B-2 (18 MG)
B-3 (2000 MG)
B-5 (5 MG)
B-6 (11 MG)
Choline (500 MG)
Inositol (1 G)
Vitamin C (250 MG)
Lotus Seed (5 G)

The Fiber/Immune Health Formula, is designed to provide the body's daily need of approximately thirty-two grams of fiber recommended by most medical professionals. Promoting digestive regularity and slowing the absorption of sugars, this version of the Body Aqua promotes healthy glucose levels and the growth of beneficial bacteria to improve the user's immune system. Key ingredients of the Fiber/Immune Health Formula are Vitamin A, Vitamin B1, Vitamin B2, Vitamin B6, Vitamin C, Vitamin E, Vitamin K, niacin, folate, flax seed oil, zinc, and pantothenic acid.

### Fiber Formula

Vitamin A
Vitamin C
Vitamin D
Vitamin E
Vitamin B1
Vitamin B2
Vitamin B12
Niacin
Folic acid
Panthothenic acid
Vitamin K
Fiber
Calcium
Iron
Zinc
Copper
Mangeanese
Fluoride
Iodide
molybdenum

The Teenager/Kids Essential Vitamins and Minerals Formula is developed expressly to provide the many vitamins, minerals, and nutrients that many teens and children lack in their diet. Encouraging healthy growth and abundant energy, this Body Aqua formula contains Vitamin A, Vitamin C, Vitamin D, Vitamin E, Vitamin B-1, Vitamin B-2, Vitamin B-3, Vitamin B-6, Vitamin B-9 and Vitamin B-12, folate, calcium, iron, magnesium, phosphorus, potassium, and zinc.

### Teens/Kids

Calcium (1000 mg)
Iron (8 mg)
Magnesium (180 mg)
Potassium (1600 mg)
Vitamin A (500 mg)
Vitamin B's
Vitamin C (35 mg)
Vitamin D(5 mcg)
Vitamin E (8 mg)
Zinc (5 mg)
Palate (200 mg).
   Other formulations include:

### Relaxation/Stress Relief Formula

Vitamin A—(15,000 IU)
Vitamin B1—(50 MG)
Vitamin B3—(50 MG)
Vitamin B6—(100 MG)
Vitamin B12—(100 MCG)
Vitamin C—(250 MG)
Vitamin E—(200 IU)
Panthothenic acid
Choline
Inositol
Calcium—(800 MG)
Chromium—(50 MG)
Magnesium—(400 MG)
Zinc
Panax ginseng—(800 MG)
Siberian ginseng—(800 MG)
Valerian
Kava Kava
   Another formulation is:

### Enhancement Drive Formula

Ginseng (250 mg)
Homey Goat weed (50 mg)
Kava (250 mg)
Mac (Perurian ginseng) (50 mg)
L-Arginine (150 mg)
Of deer antler velvet (150 mg)
Yohimbe (150 mg)
Fruit Lychii (Lycium barbarum) (150 mg)
B-Complex (50 mg)
Vitamin A (50 mg)
Vitamin E (50 mg)
(He Ho) Oh Su (Polygonum multiflorum) (50 mg)
Zinc (50 mg)
Damiana (Turnora aphrodi) (50 mg)
Fenugreek (Trigonell foenum-graceum) (50 mg)

The Body Aqua is packaged in an approximately sixteen ounce water bottle which is manufactured of lightweight, recyclable plastic material. The front of the unit features the Body Aqua logo, an illustrative outline of the human body. The back of the bottle features an ingredient and nutrient listing. The Body Aqua can be sold individually or offered for sale in twelve and twenty-four count packages.

Using the Body Aqua is very simple and straightforward. First, the user purchases bottles of the Body Aqua in accordance to the deired formula. A homemaker may purchase the All Essential Vitamin/Mineral Formula in the male and female version for consumption by her husband and herself, as well as the Teenager/Kids version for her children. A person who is watching their weight and diet may purchase the Weight Control/Belly Fat Formula in the pear or strawberry flavor. The Body Aqua is served over ice or chilled in the refrigerator and consumed as desired, providing a delightful and nutritious energy boost throughout the day. Body Aqua provides consumers with a simple and convenient means of acquiring the vitamins and minerals they need on a daily basis. A delicious line of bottled water comprised of low sodium, naturally sweetened formulas in a myriad of flavors, the Body Aqua distributes micro nutrients through the body, simply by enjoying a refreshing drink. Drinking Body Aqua eliminates the need to purchase and keep track of a vast collection of all necessary vitamins. A practical alternative to taking an arsenal of hard-to-swallow and unpleasant tasting pills, the Body Aqua provides all of the vitamins and minerals needed in one, easily consumed and delicious flavored water. Adults and children who are reluctant to take orally ingested vitamins easily can drink and enjoy the Body Aqua.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A nutritionally fortified, sugar free, low calorie, carbohydrate free, and low sodium water beverage, comprising nutritional components.

2. The beverage of claim 1, formulated for cardio, energy, igniter fuel comprising: 3 Grams Glutamine, 10 mg alanine, 10 g arginine, 4.8 g branch chain amino acids, 2000 mg vitamin C, 100 mg vitamin B complex. 100 mcg biotin, 15 mg iron, 400 mg magnesium, 0.5 mg zinc, 800 mg ginseng.

3. The beverage of claim 1 formulated for skin and wrinkles comprising: 100 mg vitamin B complex, 1 mg pyridoxine, 30 mcg biotin, 5000 IU•vitamin A, 20 mcg vitamin K, 200 IU vitamin E, 250 mg vitamin C, 1.5 mg copper, 0.5 mg zinc, 200 mg dimethylaminoethanol, 50 mg chromioum, 20 mg alpha-liopic acid, 25 mg hyaloronic acid.

4. The beverage of claim 1 formulated for joints and cartilage comprising: 500 mg proline, 2.5 g glucosamine, 3 g fish oil, 1 g methyl sulfonylmethone, 5000 IU vitamin A, 100 mg vitamin B, 250 mg vitamin C, 400 IU vitamin D, 400 mg manganese, 1.5 g omega 3 fatty acids.

5. The beverage of claim 1 formulated for essential vitamins and minerals for men comprising: 15000 IU vitamin A, 250 mg vitamin C. 200 IU vitamin E, 50 mg vitamin B, 18 mg riboflavin, 1000 mg niacin, 11 mg pyridoxine, 800 mg calcium, 800 mg phosphorous, 400 mg magnesium, 05. mg zinc.

6. The beverage of claim 1 formulated for essential vitamins and minerals for women comprising: 15 000 IU vitamin A, 250 mg vitamin C, 200 IU vitamin E, 400 IU vitamin D, 200 mg vitamin B-6, 100 mcg B-12, 600 mcg folic acid, 800 mg calcium, 5 mg boron, 400 mg magnesium, 20 mcg vitamin K.

7. The beverage of claim 1 formulated for essential vitamins and minerals for teenagers and children comprising:
1000 mg calcium, 8 mg iron, 180 mg magnesium, 1600 mg potassium, 500 mg vitamin A, vitamin B, 36 mcg vitamin C, 5 mcg vitamin D, 8 mg vitamin E, 5 mg zinc, 200 mg folate.

8. The beverage of claim 1 formulated for weight control and belly fat comprising: 18 mg B-2, 2000 mg B-3, 5 mg B-5, 11 mg B-6, 500 mg choline, 1 g inositol, 250 mg vitamin C, 5 g lotus seed.

9. The beverage of claim 1 formulated for fiber comprising:
vitamin A, vitamin C, vitamin D, vitamin E, vitamin B1, vitamin B2, vitamin B12, niacin, folic acid, pantothenic acid, vitamin K, fiber, calcium, iron, zinc, copper, manganese, fluoride, iodide, molybdenum.

10. The beverage of claim 1 formulated for relaxation and stress relief comprising: 15,000 vitamin A, 50 mg vitamin B1, 50 mg vitamin B3, 100 mg vitamin B6, 100 mcg vitamin B12, 250 mg vitamin C, 200 IU vitamin E, pantothenic acid, choline, inositol, 800 mg calcium, 50 mg chromium, 400 mg magnesium, zinc, 800 mg panax ginseng, 800 mg Siberian ginseng, valerian, kava kava.

11. The beverage of claim 1 formulated for enhancement drive comprising: 250 mg ginseng, 50 mg horney goat weed, 250 kava, 50 mg Mac (Peruvian ginseng), 150 mg L-arginine, 150 mg deer antler velvet, 150 yohimbe, 150 mg fruit lychii, (Lycium barbarum), 50 mg B-complex, 50 mg vitamin A, 50 mg vitamin E, 50 mg (He Ho)Oh Su (Polygonum multiflorum), 50 mg zinc. 50 mg damiana (Turnora aphrodi), 50 mg fenugreek (trigonell foenum-graceurn).
